(19) United States
(12) Patent Application Publication
(10) Pub. No.: US 2002/0198119 A1
(43) Pub. Date: Dec. 26, 2002

(54) Powdered soap

(76) Inventor: Edmund D. George, Warwick, RI (US)

Correspondence Address: Dike, Bronstein, Roberts & Cushman, Intellectual Property Practice Group, Edwards & Angell, LLP, P. O. Box 9169, Boston, MA, 02209, US

(21) Appl. No.: 10/161,204

(22) Filed: Jun. 3, 2002

Related U.S. Application Data
(60) Provisional application No. 60/297,319, filed on Jun. 11, 2001.

(51) Int. Cl. A61K 7/50

(52) U.S. Cl. 510/130; 510/446; 510/481

(57) ABSTRACT

An improved powdered soap is disclosed. The powdered soap has a composition that includes a surfactant/soap system, which should comprise in the range of about 64% to 99% of the powdered soap composition, and an anti-itch/anti-dryness ingredient. Optionally, but preferably, the powdered soap includes at least one flow agent and one or more of each of a fragrance, a colorant, a foam booster, and an exfoliant.

### CROSS REFERENCE TO RELATED APPLICATION

This application claims priority from, and incorporates by reference the entirety of U.S. Provisional Patent Application Serial No. 60/297,319, which was filed on Jun. 11, 2001.

### BACKGROUND OF THE INVENTION

1. Field of the Invention.

The present invention relates generally to cleansing products and, more particularly, to powdered soaps that include ingredients for improving the tactile effects produced by use of the soap without detracting from the soap's cleansing prowess.

2. Background

Few can debate the utility of washing one's hands with soap, a practice which medical experts have long recognized as being one of, if not the most simple yet effective methods of ridding one's hands of dirt, germs and bacteria.

Most individuals aspire to wash their hands regularly, but find that doing so often has adverse consequences that, in their opinion, outweigh the utility of frequent hand washing. Among the reasons most often cited as explanations for why people are dissuaded from hand washing are that the process renders their hands dry and itchy to the touch, as well as red in appearance, and that the soap leaves an unpleasant odor on their hands.

A complaint that is unique to bar-type soaps is that after only a few hand washings, bar soaps often become water-logged, which, in turn, renders them tactilely unpleasant. Also, people are deterred from using bar soaps in public areas because they are uncomfortable about who may have already used that particular soap bar.

Among the alternatives to bar soaps are soaps that are powder in form. Generally, the majority of such powdered soaps are placed into containers in high-traffic areas (e.g., public rest rooms, and rest rooms/kitchens located in businesses and restaurants) where the soap is dispensed on a use-by-use basis to hand washers, who then simply wet the soap with water to wash their hands.

Only a small amount of the powdered soap is dispensed to each user, with the remainder of the soap being housed in the container. This use-by-use dispensing system is advantageous because it allows powdered soaps to avoid the aforementioned problems associated with bar-type soaps.

Unfortunately, powdered soaps suffer from several disadvantages, mostly owing to their powdered form. For example, people frequently complain that powdered soaps are not especially “lather-building,” which, in turn, hinders the process of massaging the soap around the periphery of their hands. Moreover, users also complain that powdered soaps have a somewhat “detergent-like” odor when dry, and an unpleasant, almost rotten odor when wet. Further, people who use powdered soaps almost universally proclaim them to be especially tactilely harsh as compared to other soaps.

Therefore, a need exists for a powdered soap that exhibits excellent cleansing prowess, but that, as compared to known powdered soaps, is more lather-building, has a more pleasant scent, and is less tactilely harsh to a user's skin.

### SUMMARY OF THE INVENTION

These and other needs are met by the present invention, which provides a free-flowing powdered soap comprised of a surfactant/soap system and an itch inhibitor. Generally, the powdered soap composition further comprises at least one flow agent, and one or more of each of a colorant, a fragrance, an exfoliant, and a foam/lather booster.

The following table illustrates some of the potential ingredients of a powdered soap according to the present invention, and the respective weight percentage options/ranges (with respect to the overall composition of the soap) for each ingredient.

In an exemplary aspect of the present invention, the bulk of the composition (e.g., in the range of about 64% to 99% by weight) of the powdered soap comprises a surfactant/soap system. Preferably, this system includes a blend of two or more individual surfactants/soaps in order to gain the benefits (e.g., cleansing prowess, increased foam/lather generation, more enduring foam/lather, etc.) of each surfactant/soap.

The powdered soap also includes at least one ingredient that is effective to inhibit skin itchiness and dryness following use of the soap, e.g., for hand washing. Generally, this “anti-itch” ingredient comprises in the range of about 1% to 20% by weight of the overall composition of the powdered soap.

Optionally, but preferably, the powdered soap also includes at least one flow/dispersion agent in order to facilitate dispensing of the soap, and to prevent the soap from caking when wet (i.e., to aid in the “smoothing” of the soap). However many flow agents are included in the soap, it/they should comprise, in total, in the range of about 0.5% to 12.0% by weight of the overall composition of the powdered soap.

In a preferred aspect of the present invention, the powdered soap includes a plurality of (preferably three) flow agents, with three distinct flow agents being currently preferred.

Generally, each flow agent comprises in the range of about 0% to 8% of the overall composition of the powdered soap.

Optionally, but preferably, a powdered soap in accordance with the present invention also includes one or more fragrances or perfumes to provide the soap with a pleasant odor during and after use. When present, such fragrance(s) generally account(s) for up to about 2% by weight of the overall composition of the soap.

Additionally, a powdered soap of the present invention may include one or more exfoliants such that use of the soap promotes the removal of dead skin and encourages skin renewal.

Optionally, a powdered soap of the present invention may also include one or more foam/lather boosters that promote the accumulation of lather/foam upon the massaging of the wet powdered soap into a user's hands.

A powdered soap in accordance with the present invention may also be treated, as is generally known in the art, with one or more colorants to provide the powdered soap with a desired color. Exemplary colorants are dyes, and/or pigments, and/or colors that are either certified by the Food and Drug Administration (FDA), non-certified by the FDA, or not subject to certification by the FDA.

### DETAILED DESCRIPTION OF THE INVENTION

In an exemplary embodiment of the present invention, a free-flowing powdered soap is comprised of a surfactant/soap system and an itch inhibitor. Generally, the powdered soap composition further comprises at least one flow agent, and one or more of each of a colorant, a fragrance, an exfoliant, and a foam/lather booster.

The following table illustrates some of the potential ingredients of a powdered soap according to the present invention, and approximate weight percentage options/ranges (with respect to the overall composition of the soap) for each ingredient.

TABLE

| | Range of | Range of | Currently Most |
|---|---|---|---|
| | Acceptable | Preferred | Preferred |
| | Weight | Weight | Weight |
| Ingredient | Percentages | Percentages | Percentage |
| Surfactant/Soap | 64%-99% | 76.5%-88% | 82% |
| System | | | |
| Anti-itch/Anti- | 1%-20% | 8%-12% | 10% |
| dryness | | | |
| ingredient(s) | | | |
| First Flow Agent | 0%-2% | 0.5%-1.25% | 1% |
| Second Flow | 0%-4% | 0.75%-3% | 2% |
| Agent | | | |
| Third Flow | 0%-8% | 2.25%-6% | 4% |
| Agent | | | |
| Fragrance(s) | 0%-2% | 0.5%-1.25% | 1% |

As indicated in this Table, the bulk of the composition of the powdered soap comprises a surfactant/soap system. Preferably, this system includes a blend of two or more individual surfactants/soaps in order to gain the benefits of each surfactant/soap.

However many surfactant(s)/soap(s) actually comprise the surfactant/soap system, it/they should comprise, in total, in the range of about 64% to 99% by weight of the overall composition of the powdered soap, with a range of about 76.5% to 88% by weight being preferred.

Numerous surfactants and soaps can be included as part of the surfactant/soap system. Among the suitable surfactants/soaps are sodium or potassium salts (or blends of sodium and potassium salts) of triglycerides and/or fatty acids. Exemplary trigylcerides and fatty acids include, but are not limited to, palm oil coconut oil, palm kernel oil, and beef tallow.

Currently, it is preferred for the surfactant/soap system of the powdered soap to be comprised of a blend of two sodium salts of triglycerides, wherein the blend comprises about 82% by weight of the overall composition of the powdered soap.

Two currently preferred sodium salts are sodium palmate (i.e., a sodium salt of palm oil) and sodium cocoate powder (i.e., a sodium salt of coconut oil). A blend of these sodium salts not only provides the soap with excellent cleansing prowess, but also with an increased rate of foam/lather generation (due to the inclusion of sodium cocoate powder), and a more enduring foam/lather (due to the inclusion of sodium palmate).

The powdered soap also includes at least one ingredient that is effective to inhibit skin itchiness and dryness following use of the soap, e.g., for hand washing.

As illustrated in the above Table, such an ingredient comprises in the range of about 1% to 20% by weight of the overall composition of the powdered soap, with a range of about 8% to 12% being preferred. Currently, it is most preferred for this ingredient to comprise about 10% by weight of the overall composition of the powdered soap.

An exemplary anti-itch/anti-dryness ingredient is colloidal oatmeal or oat flour.

Optionally, but preferably, the powdered soap also includes at least one flow/dispersion agent in order to facilitate dispensing of the soap, and to prevent the soap from caking when wet (i.e., to aid in the “smoothing” of the soap). However many flow agents are included in the soap, it/they should comprise, in total, in the range of about 0.5% to 12.0% by weight of the overall composition of the powdered soap, with a range of about 3.5% to 10.25% by weight being preferred.

In a preferred embodiment of the present invention, the powdered soap includes a plurality of flow agents, with three distinct flow agents being a currently most preferred number. Among the suitable flow agents are silica, tapioca powder, and aluminum starch octenyl succinate.

In an exemplary embodiment of the present invention, silica, aluminum starch octenyl succinate, and tapioca powder are all included as flow agents in the powdered soap. In such an embodiment, the weight percentages of silica, aluminum starch octenyl succinate, and tapioca powder comprise, respectively, in the range of about 0% to 2% (preferably about 0.5% to 1.25%, currently most preferably about 1%), about 0% to 4% (preferably about 0.75% to 3%, currently most preferably about 2%), and about 0% to 8% (preferably about 2.25% to 6%, currently most preferably about 4%) of the overall composition of the powdered soap.

Optionally, but preferably, a powdered soap in accordance with the present invention also includes one or more fragrances or perfumes to provide the soap with a pleasant odor during and after use. When present, such fragrance(s) account(s) for up to about 2% (preferably in the range of about 0.5% to 1.25 percent, currently most preferably about 1%) by weight of the overall composition of the soap.

Examples of suitable fragrances include, but are not limited to, floral, citrus, fruit, and botanical fragrances, and essential oils such as patchouli, peppermint, chamomile, and eucalyptus.

Additionally, a powdered soap of the present invention may include one or more exfoliants such that use of the soap promotes the removal of dead skin and encourages skin renewal.

Exemplary exfoliants include, but are not limited to, loofah, apricot seeds, bran, jojoba beads, and polymer beads (e.g., beads of polyethylene, polypropylene and nylon.

Optionally, a powdered soap of the present invention may also include one or more foam/lather boosters that promote the accumulation of lather/foam upon the massaging of the wet powdered soap into a user's hands.

Exemplary foam boosters include, but are not limited to, synthetic detergents (e.g., sodium cocoyl isethionate).

A powdered soap in accordance with the present invention may also be treated, as is generally known in the art, with one or more colorants to provide the powdered soap with a desired color. Exemplary colorants are dyes, and/or pigments, and/or colors that are either certified by the Food and Drug Administration (FDA), non-certified by the FDA, or not subject to certification by the FDA.

In an embodiment in which one or more exfoliants, lather/foam boosters, and/or colorants comprise part of the overall composition of the powdered soap, the weight percentages indicated in the Table above are adjusted (i.e., reallocated) to allow for the inclusion this/these extra ingredients.

This may be accomplished by renormalizing the total composition of the powdered soap. Alternatively, the weight percentage of soap/surfactant system can be reduced pro rata to account for the additional weight percentage of this/these other ingredient(s). As noted in the Table above, however, the weight percentage of the surfactant/soap system should not be less than about 64% of the overall composition of the powdered soap, so as not to compromise the powdered soap's cleansing prowess.

In addition to the ingredients noted above for the powdered soap, other exemplary ingredients (in lieu of, and/or in addition to, any of the triglycerides, fatty acids, surfactants/soaps, anti-itch/anti-dryness ingredients, flow agents, exfoliants, colorants, fragrances, and foam boosters listed above) may include those listed/referenced in the Cosmetic, Toiletry, and Fragrance Association (CTFA) International Cosmetic Ingredient Dictionary and Handbook, McCutcheon's Volume 1 (Emulsifiers & Detergents), McCutcheon's Volume 2 (Functional Materials), and/or other comparable sources known to the ordinarily skilled artisan.

The foregoing description of the invention is merely illustrative thereof, and it is understood that variations and modifications can be effected without departing from the scope or spirit of the invention as set forth in the following claims. All publications referenced herein are incorporated by reference in their entirety.

What is claimed is:

1. A powdered soap having an overall composition, the powdered soap comprising:
a surfactant system comprising in the range of about 64% to 99% by weight of the overall composition of the powdered soap; and
an anti-itch ingredient.

2. The powdered soap of claim 1, further comprising:
at least one flow agent, each flow agent comprising at least 0.5% by weight of the overall composition of the powdered soap.

3. The powdered soap of claim 2, wherein the powdered soap includes three flow agents, and wherein the three flow agents comprise, in total, in the range of about 3.5% to 10.25% by weight of the overall composition of the powdered soap.

4. The powdered soap of claim 2, wherein each of the at least one flow agent is selected from the group consisting of silica, tapioca powder, and aluminum starch octenyl powder.

5. The powdered soap of claim 1, further comprising:
at least one additional additive, wherein each additive is selected from the group consisting of exfoliants, foam boosters, fragrances, and colorants.

6. The powdered soap of claim 1, wherein the anti-itch ingredient comprises in the range of about 1% to 20% by weight of the overall composition of the powdered soap.

7. The powdered soap of claim 1, wherein the surfactant system is a sodium salt of a triglyceride.

8. The powdered soap of claim 1, wherein the surfactant system is a sodium salt of a fatty acid.

9. The powdered soap of claim 1, wherein the surfactant system is comprised of a blend of sodium palmate and sodium cocoate powder.

10. The powdered soap of claim 1, wherein the anti-itch ingredient is colloidal oatmeal.

11. The powdered soap of claim 1, wherein the surfactant system comprises about 76.5% to 88% by weight of the overall composition of the powdered soap.

12. A powdered soap having an overall composition, the powdered soap comprising:
a surfactant system comprising at least 64% by weight of the overall composition of the powdered soap;
an anti-itch ingredient comprising at least 1% by weight of the overall composition of the powdered soap; and
at least one flow agent, wherein each flow agent comprises at least 0.5% by weight of the overall composition of the powdered soap.

13. The powdered soap of claim 12, further comprising:
at least one additional additive, wherein each additive is selected from the group consisting of exfoliants, foam boosters, fragrances, and colorants.

14. The powdered soap of claim 12, wherein the powdered soap includes three flow agents, and wherein the three flow agents comprise, in total, in the range of about 3.5% to 10.25% by weight of the overall composition of the powdered soap.

15. The powdered soap of claim 12, wherein each of the at least one flow agent is selected from the group consisting of silica, tapioca powder, and aluminum starch octenyl powder.

16. A powdered soap having an overall composition, the powdered soap comprising:
a surfactant system comprising at least 64% by weight of the overall composition of the powdered soap;
an anti-itch ingredient comprising in the range of about 1% to 20% by weight of the overall composition of the powdered soap; and
a plurality of flow agents, wherein each flow agent comprises at least 0.5% by weight of the overall composition of the powdered soap, and wherein the plurality flow agents comprise, in total, in the range of about 3.5% to 10.25% by weight of the overall composition of the powdered soap.

17. The powdered soap of claim 16, further comprising:
at least one additional additive, wherein each additive is selected from the group consisting of exfoliants, foam boosters, fragrances, and colorants.
